(19) United States
(12) Patent Application Publication
(10) Pub. No.: US 2025/0381074 A1
(43) Pub. Date: Dec. 18, 2025

(54) NASAL PASSAGE INSERT AND PACKAGING ASSEMBLY THEREFOR

(71) Applicant: Devin M. Yaeger, Los Angeles, CA (US)

(72) Inventor: Devin M. Yaeger, Los Angeles, CA (US)

(21) Appl. No.: 19/236,155

(22) Filed: Jun. 12, 2025

Related U.S. Application Data
(60) Provisional application No. 63/659,236, filed on Jun. 12, 2024.

(51) Int. Cl. A61F 13/20; A61K 9/00; A61K 45/06

(52) U.S. Cl. CPC A61F 13/2005; A61K 9/0043; A61K 45/06; A61M 15/08; A61M 31/002

(57) ABSTRACT

A nasal passage insert packaging assembly that includes at least a first insert member that is configured to be received in a first nasal passage. The first insert member is made of an absorbent material and includes a medicinal substance therein. The assembly includes a container that defines a container interior. The first insert member is disposed in the container interior and the container is scaled.

### CROSS-REFERENCE TO RELATED APPLICATIONS

This application claims the benefit of U.S. Provisional Application No. 63/659,236, filed on Jun. 12, 2024, the entirety of which is incorporated by reference herein.

### FIELD OF THE INVENTION

The present invention relates to an orifice or nasal passage insert or plug and packaging assembly.

### BACKGROUND OF THE INVENTION

Nasal passages or other body orifices may benefit from inserts, plugs or other members that can be inserted for protection or for delivery of pharmaceuticals and the like. Swabs may be used currently. However, swabs or only inserted and then removed and are not left in the nasal passage for a period of time to allow the medicinal substance thereon to be properly delivered through the inner surface of the nasal passage. The present invention addresses some or all of these issues, as discussed below.

The background description disclosed anywhere in this patent application includes information that may be useful in understanding the present invention. It is not an admission that any of the information provided herein is prior art or relevant to the presently claimed invention, or that any publication specifically or implicitly referenced is prior art.

### SUMMARY OF THE PREFERRED EMBODIMENTS

In accordance with a first aspect of the present invention there is provided a nasal passage insert packaging assembly that includes at least a first insert member that is configured to be received in a first nasal passage. The first insert member is made of an absorbent material and includes a medicinal substance therein. The assembly includes a container that defines a container interior. The first insert member is disposed in the container interior and the container is sealed. The first insert member includes a main body portion that defines a first axis and has a generally cylindrical shape. At least a first tunnel is defined through the main body portion between a top surface and a bottom surface of the main body portion. The first tunnel may extend axially through the main body portion. A second tunnel may be defined through the main body portion between the top surface and the bottom surface of the main body portion and the second tunnel may be offset from the first axis.

The first tunnel includes an inner surface and the medicinal substance may be included on the inner surface. A different type of substance (a second medicinal substance) may be in the tunnel. This substance may be delivered via inhalation. A second insert member may be included in the package. The insert member may also include a retrieval features extending from an end thereof.

The present invention may also include a method for intranasal delivery of a medicinal substance. The method may include obtaining a sealed container that includes an insert member in a container interior thereof, where the first insert member comprises an absorbent material and includes a medicinal substance contained therein, opening the package to expose the insert member, where the insert member is at least partially saturated with the medicinal substance, inserting the insert member into a nostril of a user such that the plug contacts an inner surface of the nasal cavity, allowing the insert member to remain in the nostril for a predetermined period of time sufficient to permit at least a portion of the medicinal substance to be transferred to the inner surface of the nasal cavity; and removing the insert member from the nostril after the predetermined period of time. The predetermined period of time is more than thirty seconds.

The present invention is directed to disposal single use human orifice or nasal insert assembly. In a preferred embodiment, the inserts may be made of a semi-permeable material, e.g., silicone, polyurethane, thermoplastics, plastic, nylon, cotton, compressed cellulose, gauze or other natural fiber or a synthetic material with pores or openings in it so that the medicinal substance can soak into or be contained within the insert. Foam, rubber or the like can also be used. The inserts are shaped to fit snuggly in a human's nostril(s) (or ears). In a preferred embodiment, the plugs include thereon, therein or are soaked in or at least partially saturated with a medicinal liquid, such as menthol, eucalyptus oil, a Neosporin or Vaseline type ointment, a prescription medication, etc. . . . .

The present disclosure relates to devices and methods for intranasal delivery of a medicinal substance. More specifically, the disclosure pertains to a plug or insert that is configured for temporary placement within a nasal cavity to facilitate localized and/or systemic administration of one or more active agents via the mucosal surfaces of the nostril.

In one embodiment, the device comprises a plug, insert, or other conformable structure dimensioned for insertion into the anterior portion of a human nostril. The plug may be formed from a biocompatible material such as foam, silicone, hydrogel, or other elastomeric or absorbent substrate. The material may be resiliently compressible to permit comfortable retention within the nasal passage during use.

At least a portion of the outer surface of the plug is coated with or impregnated with a medicinal substance (which may be referred to herein as a medicinal substance). The medicinal substance may be present in liquid, gel, powder, or microencapsulated form, and may include, without limitation, decongestants, antihistamines, corticosteroids, numbing agents or balms, soothing agents or balms, vasodilators, peptides, or other therapeutic or prophylactic agents suitable for intranasal administration.

In some embodiments, the medicinal substance is configured to be released upon contact with the mucosal surface of the nasal passage. The device is intended to remain in place for a predetermined period of time, sufficient to allow transfer of the active agent to the mucosal lining of the nasal cavity. Upon completion of the delivery period, the plug may be manually removed and discarded or retained for repeated use, depending on whether the embodiment is disposable or reusable.

The geometry of the plug may be generally cylindrical, conical, bulbous, or anatomically contoured to enhance comfort, retention, and effective surface contact. All of the shapes in the previous sentence are considered “generally cylindrical” as used herein. In certain embodiments, the plug may include a retrieval feature, such as a stem, tab, or string, to facilitate removal from the nostril after use.

Optionally, the plug may further include one or more structural features to control the rate of drug release, promote adhesion to the mucosa, or resist undesired movement within the nasal cavity. In some embodiments, the plug may also be configured to occlude the nostril partially or completely to increase contact time and reduce evaporative loss of the medicinal substance.

In some embodiments, the plug or insert includes one or more air passage(s), breathing channel(s), tunnel(s) or lumen(s) extending through at least a portion of the plug. This tunnel permits airflow through the nostril while the plug is in place, thereby allowing the user to breathe nasally during administration of the medicinal substance. The cross-sectional area and length of the air tunnel may be selected to balance airflow capacity with structural integrity and drug-delivery surface area. The tunnels may be central and extend along the longitudinal axis of the plug or the main body portion thereof or one or more tunnels may be offset from the longitudinal axis. The tunnels may also extend diagonally or non-parallel to the central axis. In some implementations, the inner surface of the tunnel may also be coated with the medicinal substance to further enhance delivery during inhalation. One or more tunnels or channels that are open to the outside, side wall of the main body portion may also be defined in the outside surface.

In a preferred embodiment, one or more inserts or plugs (preferably two) are housed within a sealed package or container that contains a measured quantity of the medicinal substance. The packaging assembly may include a primary compartment or cavity that securely holds the plug and a separate reservoir or integrated portion containing the medicinal substance in liquid or semi-liquid form. The packaging may be configured such that the plug is either pre-soaked in the substance during storage or comes into contact with the substance upon user activation, such as by rupturing an internal barrier, squeezing the package, or opening a sealed compartment. Generally, the invention may provide an inexpensive and simple package plus a cylindrical applicator or insert member plus a medicinal substance or saturation agent all combined for quick and easy use on the go, e.g., by someone that is sick or possibly for inter nasal drug users after a long night of partying.

In one example, the packaging may take the form of a flexible blister pouch or thermoformed tray with an inner liner coated with or filled with the medicinal substance. The packaging may include frayed or jagged edges for ease of opening, i.e., easy to tear it open and remove the wet insert member and insert it in into a nostril for a few minutes or hours. The seal may be a reusable seal. Upon opening the package, the plug is already saturated with the substance and ready for immediate use. Alternatively, the packaging may include a frangible seal between two chambers, allowing the user to activate the soaking process immediately prior to use by pressing or twisting to mix the plug with the medicinal solution.

### BRIEF DESCRIPTION OF THE DRAWINGS

FIG. 1 is a perspective view of a nasal passage insert packaging assembly in accordance with a preferred embodiment of the present invention;

FIG. 2 is a perspective view of an insert member;

FIG. 3 is a cross-section taken along line 3-3 of FIG. 2; and

FIG. 4 is a perspective view of an insert member inserted into a nasal passage.

### DETAILED DESCRIPTION OF THE PREFERRED EMBODIMENTS

The following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be, but not necessarily are references to the same embodiment; and, such references mean at least one of the embodiments. If a component is not shown in a drawing then this provides support for a negative limitation in the claims stating that that component is “not” present. However, the above statement is not limiting and in another embodiment, the missing component can be included in a claimed embodiment.

Reference in this specification to “one embodiment,” “an embodiment,” “a preferred embodiment” or any other phrase mentioning the word “embodiment” means that a particular feature, structure, or characteristic described in connection with the embodiment is included in at least one embodiment of the-disclosure and also means that any particular feature, structure, or characteristic described in connection with one embodiment can be included in any embodiment or can be omitted or excluded from any embodiment. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others and may be omitted from any embodiment. Furthermore, any particular feature, structure, or characteristic described herein may be optional. Similarly, various requirements are described which may be requirements for some embodiments but not other embodiments. Where appropriate any of the features discussed herein in relation to one aspect or embodiment of the invention may be applied to another aspect or embodiment of the invention. Similarly, where appropriate any of the features discussed herein in relation to one aspect or embodiment of the invention may be optional with respect to and/or omitted from that aspect or embodiment of the invention or any other aspect or embodiment of the invention discussed or disclosed herein.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted.

It will be appreciated that the same thing can be said in more than one way. Consequently, alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long,” “up,” “down,” “aft,” “forward,” “inboard,” “outboard” and “below” used herein are merely for ease of description and refer to the orientation of the components as shown in the figures. It should be understood that any orientation of the components described herein is within the scope of the present invention.

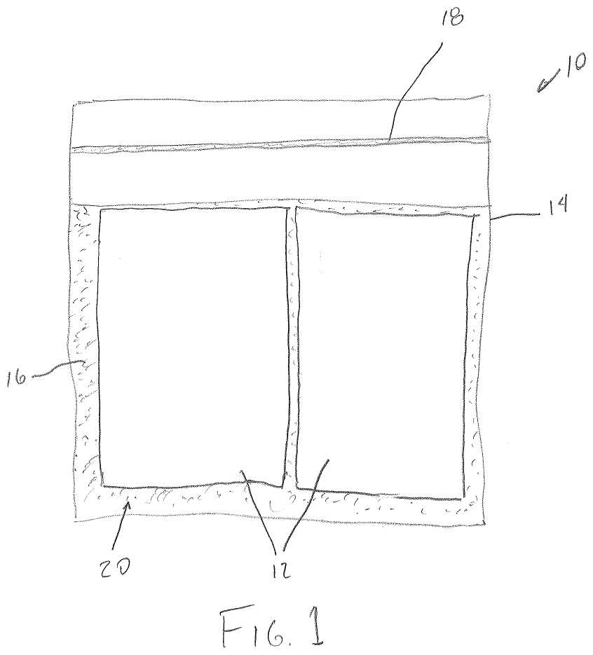

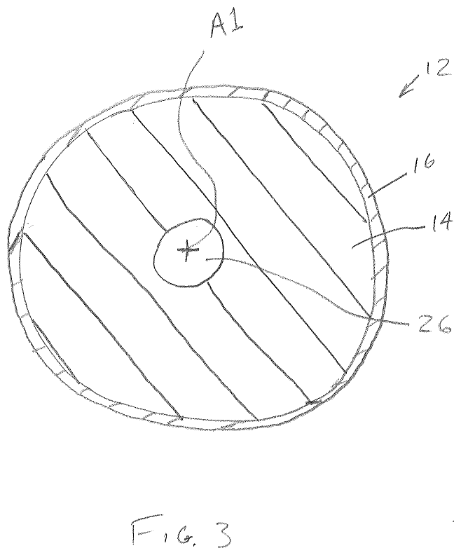

As shown in FIGS. 1-4, the present invention is directed to a nasal passage insert packaging assembly 10 that includes one or more plugs or insert members 12 that are configured to be received in nasal passages. The insert members 12 comprise an absorbent material and includes a medicinal substance 16 thereon and/or therein. See the stippling in FIG. 1 representing the medicinal substance 16 in the container 14 and the coating of medicinal substance 16 in FIG. 3. The coating concept is not limiting and it will be appreciated that the medicinal substance will be present on the outer surface of the main body portion 22 of the insert members and will be absorbed into the pores thereof as well. The packaging assembly 10 includes a container 14 that defines a container interior 20 and the insert members 12 are disposed in the container interior 20. The container 14 is sealed. See, e.g., seal 18 in FIG. 1. It will be appreciated that the container 14 comprises a transparent or see through material in FIG. 1 so the insert members 12 can be seen through the packaging. This is not a limitation. The container also includes a flap where the seal is located. This is also not a limitation.

As shown in FIG. 1, the pair of insert members 12 are preferably disposed in a sealed in the container 14, bag or other package, which may comprise a plastic, foil or similar material. The material of the container may be rigid or may be a movable, bendable or foldable plastic or foil. The container is preferably filled with the medication, liquid, viscous, ointment, etc. that the insert members are meant to deliver. Non-limiting dimensions for the container 14 may include about 1.5 inches to about 2 inches in length, about 1.5 inches to about 2 inches in width and about 0.4 inches to about 0.5 inches in thickness. The package or container may be sealed or hermetically sealed. In use, the insert members 12 may be positioned in the nostrils for several minutes to several hours (any predetermined time) to provide relief to the user or to deliver the substance, coating, agent, medicinal treatment, pharmaceutical or the like. The insert members 12 allow the medicinal substance 16 to remain in the nasal passage for a time longer than if the user inserts a swab or the like.

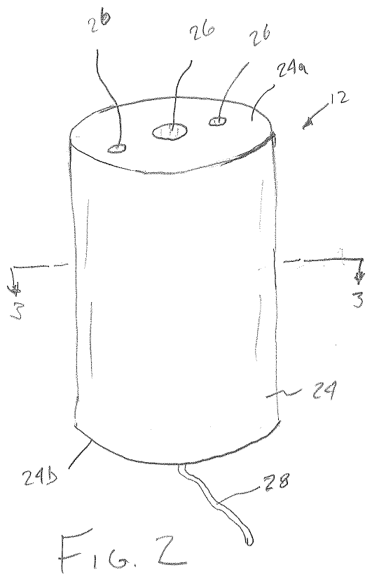

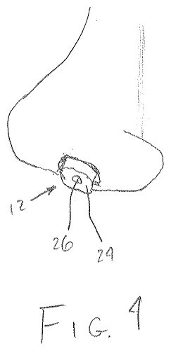

In use, the plugs 12 provide continuous delivery of the substance thereon and, due to the snug fit in the nostrils, apply pressure and thus more directly administer the agent or substance in the nose. As shown in FIG. 2, each plug 12 includes a main body portion 24. The main body portion 24 may include one or more tunnels 26, passages, through holes or other openings defined therein or therethrough that provide(s) air to pass therethrough so that the user can breathe through the nose (through the tunnel) while utilizing the plugs. The tunnel 26 may be defined axially through the plug (see axis Al in FIG. 3). The tunnel extends through the main body portion 24 from the top surface 24a to the bottom surface 24b. A plurality of tunnels 26 may be defined through the main body portion 24, as shown in FIG. 2 (which includes three tunnels). The tunnels may be various sizes (smaller and larger). In another embodiment, the tunnel may be omitted and the user may utilize one plug or one plug at a time in opposite nostrils. FIG. 4 shows an insert member 12 inserted into a nasal passage.

The insert 12 may be formed from an absorbent, hydrophilic, porous or sponge-like and biocompatible material, such as open-cell foam, hydrogel, or a porous polymer, configured to retain a volume of a medicinal substance upon contact. The material may be referred to generally as hydrophilic and include any of the properties discussed herein. In certain embodiments, the material is hydrophilic, allowing the insert 12 to soak up aqueous or gel-based formulations efficiently. A hydrophilic matrix configured to absorb and retain aqueous or semi-liquid medicinal substances for intranasal delivery.

It will be appreciated that one of the purposes of the present invention is to soothe or treat dry, irritated, infected or otherwise uncomfortable nose irritations, such as those experienced from exposure to high altitude, wind, allergies, dry weather conditions, medical conditions, medication side effects, nose picking, prolonged usage of insulated drugs (that lead to micro tears in the nose and general rawness), etc. The present invention may also be used as a delivery mechanism for drugs, similar to how oral lozenges are used to administer various drugs to the throat.

It will be appreciated that FIG. 1 show an exemplary size of the packaging. However, the package may be smaller relative to the plugs to form a closer fit around the nose plugs. A single plug may also be included in a package. The package interior may be at least be partially filled with the soaking medication, powder, liquid, viscous ointment, etc. that the plugs will deliver.

As shown in FIG. 2, in certain embodiments, the insert member 12 may include a retrieval feature 28, such as a stem, tab, or string, to facilitate removal from the nostril after use.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description of the Preferred Embodiments using the singular or plural number may also include the plural or singular number respectively. The word “or” in reference to a list of two or more items, covers all of the following interpretations of the word: any of the items in the list, all of the items in the list, and any combination of the items in the list.

The above-detailed description of embodiments of the disclosure is not intended to be exhaustive or to limit the teachings to the precise form disclosed above. While specific embodiments of and examples for the disclosure are described above for illustrative purposes, various equivalent modifications are possible within the scope of the disclosure, as those skilled in the relevant art will recognize. Further, any specific numbers noted herein are only examples: alternative implementations may employ differing values, measurements or ranges.

Although the operations of any method(s) disclosed or described herein either explicitly or implicitly are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

The teachings of the disclosure provided herein can be applied to other systems, not necessarily the system described above. The elements and acts of the various embodiments described above can be combined to provide further embodiments. Any measurements or dimensions described or used herein are merely exemplary and not a limitation on the present invention. Other measurements or dimensions are within the scope of the invention.

Any patents and applications and other references noted above, including any that may be listed in accompanying filing papers, are incorporated herein by reference in their entirety. Aspects of the disclosure can be modified, if necessary, to employ the systems, functions, and concepts of the various references described above to provide yet further embodiments of the disclosure.

These and other changes can be made to the disclosure in light of the above Detailed Description of the Preferred Embodiments. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features or aspects of the disclosure with which that terminology is associated. In general, the terms used in the following claims should not be construed to limit the disclosures to the specific embodiments disclosed in the specification unless the above Detailed Description of the Preferred Embodiments section explicitly defines such terms. Accordingly, the actual scope of the disclosure encompasses not only the disclosed embodiments, but also all equivalent ways of practicing or implementing the disclosure under the claims.

While certain aspects of the disclosure are presented below in certain claim forms, the inventors contemplate the various aspects of the disclosure in any number of claim forms. For example, while only one aspect of the disclosure is recited as a means-plus-function claim under 35 U.S.C. § 112, ¶6, other aspects may likewise be embodied as a means-plus-function claim, or in other forms, such as being embodied in a computer-readable medium. (Any claims intended to be treated under 35 U.S.C. § 112, ¶6 will include the words “means for”). Accordingly, the applicant reserves the right to add additional claims after filing the application to pursue such additional claim forms for other aspects of the disclosure.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. A nasal passage insert packaging assembly comprising:
at least a first insert member that is configured to be received in a first nasal passage, wherein the first insert member comprises an absorbent material and includes a medicinal substance therein,
a container that defines a container interior, wherein the first insert member is disposed in the container interior, and wherein the container is sealed.

2. The nasal passage insert packaging assembly of claim 1 wherein the first insert member includes a main body portion that defines a first axis and has a generally cylindrical shape, wherein at least a first tunnel is defined through the main body portion between a top surface and a bottom surface of the main body portion.

3. The nasal passage insert packaging assembly of claim 2 wherein the first tunnel extends axially through the main body portion.

4. The nasal passage insert packaging assembly of claim 2 wherein a second tunnel is defined through the main body portion between the top surface and the bottom surface of the main body portion.

5. The nasal passage insert packaging assembly of claim 4 wherein the second tunnel is offset from the first axis.

6. The nasal passage insert packaging assembly of claim 2 wherein the first tunnel includes an inner surface, and wherein the medicinal substance is included on the inner surface of the first tunnel.

7. The nasal passage insert packaging assembly of claim 1 further comprising a second insert member that is configured to be received in a nasal passage, wherein the second insert member includes the medicinal substance thereon.

8. The nasal passage insert packaging assembly of claim 1 wherein the first insert member comprises a hydrophilic matrix configured to absorb and retain the medicinal substance.

9. The nasal passage insert packaging assembly of claim 1 further comprising a retrieval features extending from an end thereof.

10. The nasal passage insert packaging assembly of claim 1 wherein the medicinal substance is a cold medication, flu medication, decongestant, antihistamine, corticosteroid, numbing agent, soothing agent, vasodilator or peptide.

11. A nasal passage insert packaging assembly comprising:
a first insert member that is configured to be received in a first nasal passage, wherein the first insert member includes a medicinal substance therein, wherein the first insert member includes a main body portion that defines a first axis, has a generally cylindrical shape and comprises a hydrophilic matrix configured to absorb and retain the medicinal substance, wherein at least a first tunnel is defined through the main body portion between a top surface and a bottom surface of the main body portion, wherein the first tunnel includes an inner surface, and wherein the medicinal substance is included on the inner surface of the first tunnel,
a second insert member that is configured to be received in a second nasal passage, wherein the second insert member includes a medicinal substance thereon, wherein the second insert member includes a main body portion that defines a second axis, has a generally cylindrical shape and comprises a hydrophilic matrix configured to absorb and retain the medicinal substance, wherein at least a second tunnel is defined through the main body portion between a top surface and a bottom surface of the main body portion, wherein the second tunnel includes an inner surface, and wherein the medicinal substance is included on the inner surface of the second tunnel,
a container that defines a container interior, wherein the first and second insert members are disposed in the container interior, and wherein the container is sealed, wherein a quantity of the medicinal substance is disposed in the container interior.

12. A method for intranasal delivery of a medicinal substance, comprising:
(a) obtaining a sealed container that includes an insert member in a container interior thereof, wherein the first insert member comprises an absorbent material and includes a medicinal substance contained therein;
(b) opening the package to expose the insert member, wherein the insert member is at least partially saturated with the medicinal substance;
(c) inserting the insert member into a nostril of a user such that the insert member contacts an inner surface of the nasal cavity;
(d) allowing the insert member to remain in the nostril for a predetermined period of time sufficient to permit at least a portion of the medicinal substance to be transferred to the inner surface of the nasal cavity; and
(e) removing the insert member from the nostril after the predetermined period of time.

13. The method of claim 12 wherein the predetermined period of time is more than thirty seconds.

14. The method of claim 12 wherein the first insert member includes a main body portion that defines a first axis and has a generally cylindrical shape, wherein at least a first tunnel is defined through the main body portion between a top surface and a bottom surface of the main body portion.

15. The method of claim 12 wherein the first tunnel extends axially through the main body portion.
